(19) United States
(12) Patent Application Publication
(10) Pub. No.: US 2015/0030685 A1
(43) Pub. Date: Jan. 29, 2015

(54) Sugar-Free Oral Transmucosal Fentanyl Citrate Lozenge Dosage Forms

(71) Applicants: Lynn J. Maland, Salt Lake City, UT (US); Brian I. Hague, Fredericksburg, TX (US)

(72) Inventors: Lynn J. Maland, Salt Lake City, UT (US); Brian I. Hague, Fredericksburg, TX (US)

(21) Appl. No.: 13/949,127

(22) Filed: Jul. 23, 2013

(51) Int. Cl. A61K 31/4468; A61K 9/00

(52) U.S. Cl. CPC A61K 31/4468; A61K 9/006
USPC 424/499; 514/329; 424/489

(57) ABSTRACT

A sugar-free, pharmaceutical composition comprising an oral transmucosal solid dosage form which includes an adherent carrier preblend mixture of a highly potent pharmaceutical agent, and dextrates, hydrated the composition further including a pharmaceutically acceptable sugar-free excipient.

### BACKGROUND OF THE INVENTION

1. Field of the Invention

The present invention is directed to oral, sugar-free solid dosage forms. The solid dosage forms of the invention comprise a high potency pharmaceutical agent, a suitable pharmaceutically acceptable sugar-free primary excipient, and an adherent excipient. In some instances, the solid dosage forms comprise a lozenge which includes fentanyl citrate as the high potency pharmaceutical agent, isomalt as the primary, pharmaceutically acceptable sugar-free excipient, and dextrates as the adherent excipient. The process of forming the solid dosage forms includes steps of ordered mixing, whereby the high potency pharmaceutical agent is first mixed with the dextrates to provide an adherent preblend mixture. Dextrates is defined as a mixture of saccharides (sugars) derived from the enzymatic hydrolysis of starch. The compendial form of dextrates used in the present invention is dextrates, hydrated NF. This material has a dextrose equivalent (DE) of 93%-99% meaning that it contains 93%-99% of the reducing sugar capability of an equal weight of dextrose. The seeming inconsistency of using a reducing sugar in the design of a sugar-free pharmaceutical product is part of the uniqueness of the invention. The way dextrates, hydrated is used and the properties of adherent dry powder mixtures made by combining it with a high potency active are described in detail in subsequent sections. The structural features of dextrates, hydrated and the high potency of fentanyl citrate allow for precise, predictable, and repeatable loading of the drug in the preblend, thereby requiring minimal amounts of the preblend mixture in the final solid dosage form. Accordingly, a final solid dosage form with a desired drug concentration may be provided which includes dextrates at a concentration of less than 0.5 g per serving or dosage unit, thereby qualifying the final solid dosage form as a sugar-free product. In summary, the key attributes of the present invention are (1) a dry powder adherent preblend process that exceeds the ordinary skill in the art for producing highly uniform, segregation-resistant mixtures of high potency active ingredients using a particular carrier excipient and mixing method chosen for that purpose, while avoiding the complexity and other disadvantages of traditional methods, and (2) employing the adherent preblend so made in the manufacture of a sugar-free dosage form for oral transmucosal drug delivery.

2. Background and Related Art

The most popular route of drug administration remains the use of solid or liquid formulations that are swallowed. The fate of the swallowed drug and the quality of its therapeutic effect are determined by the conditions and mechanisms it is subjected to in the gastrointestinal tract. These include exposure to gastric acid in the stomach, digestive enzymes, changing pH conditions, the level of gastrointestinal motility, the absorptive capacity of the intestinal lumen, and pre-systemic hepatic extraction. Oral transmucosal (OT) delivery, on the other hand, is a particularly advantageous delivery route for drugs that are degraded or eliminated as they pass through the gastrointestinal tract. These advantages are widely recognized in the pharmaceutical literature.

Systemic drug delivery may also be achieved through various absorptive mucosa in the body including the buccal together with the sublingual referred to as the oromucosal mucosa, as well as nasal, ocular, pulmonary, rectal, and vaginal routes of administration. For some drug molecules these non-swallowed routes of administration may provide the only therapeutically effective alternative to injection.

The basic concept of administering a drug for systemic effect via absorption from mucosal tissues is well known. William Murrell in 1879 established the use of sublingual nitroglycerin for the relief of acute anginal pain and as a prophylactic agent to be taken prior to physical exertion [The Pharmacological Basis of Therapeutics, Goodman & Gilman et al 9th ed. Ch 32, 1996.] Over time increasingly sophisticated OT dosage forms have been developed and marketed. They include different types of tablets (e.g., bioadhesive, effervescent, sublingual, fast dissolving); lozenges; lozenges-on-a-handle; laminated films; hydrogels; buccal patches; chewing gums; hollow fibers, and buccal sprays.

Among the currently available dosage forms, the only use thus far for the oromucosal lozenge on-a-handle is the fentanyl citrate product Actiq® and its generic equivalents. These fentanyl products have also been referred to as oral transmucosal fentanyl citrate or OTFC. The oromucosal lozenges currently available are unique in their size (approximately 9-10 mm in diameter and approximately 19-20 mm long) shape (right cylinder with a domed end), mass (approximately 2 g), and method of use compared to other solid OT dosage forms. These characteristics present formulation and manufacturing challenges that are very different from other solid dosage forms whether for oral or OT administration. OTFC lozenges are currently manufactured using a compressible sugar as the main carrier/bulking agent or excipient. Hence the amount of sugar that makes up the bulk of the dosage form presents considerable problems for individuals who need to limit their intake of sugar for health reasons. This is especially true for patients who require several doses per day.

Thus, while systems and methods currently exist for administering high potency pharmaceuticals via oral transmucosal delivery, important challenges remain. Accordingly, there is a need in the art for improved systems and methods to address these challenges. Such improvements are provided herein.

### BRIEF SUMMARY OF THE INVENTION

Some implementations of the present invention are directed to a sugar-free, solid oromucosal lozenge-type dosage form for the delivery of fentanyl and other high potency drugs via the mucosal membranes of the mouth. In some instances, the sugar-free solid dosage forms of the present invention comprise a sugar-free carrier particularly suited for making lozenge-type dosage units. In other instances, the present invention provides a novel combination of materials and improved methods for the manufacture of stable, segregation-resistant dry powder blends of low dosage, high potency drugs with superior content uniformity. The present invention also provides a compressed dosage unit shape having the feature of a handle that permits the patient or care giver to administer a drug in a dose-to-effect manner.

The present invention further includes various other formulations, manufacturing processes, and product performance characteristics for sugar-free OTFC dosages. It is readily apparent that the principles employed in the present invention may be beneficially applied to other high potency drugs suitable for OT administration.

These and other features of the invention will become more fully apparent in the detailed description and appended claims to follow.

### DETAILED DESCRIPTION OF THE INVENTION

It is expected that the present invention may take many other forms and shapes, hence the following disclosure is intended to be illustrative and not limiting, and the scope of the invention should be determined by reference to the appended claims.

The present invention is directed to oral, sugar-free solid dosage forms. The solid dosage forms of the present invention comprise a high potency pharmaceutical agent, a suitable pharmaceutically acceptable primary excipient, and an adherent excipient. In some instances, the solid dosage forms of the present invention comprise a lozenge which includes fentanyl citrate as the high potency pharmaceutical agent, isomalt as the primary, pharmaceutically acceptable excipient, and dextrates, hydrated as the adherent excipient. The process of forming the solid dosage forms includes steps of ordered mixing, whereby the high potency pharmaceutical agent is first mixed with the dextrates, hydrated to provide an adherent preblend mixture. The structural features of spray dried dextrates, hydrated and the high potency of fentanyl citrate allow for predictable and repeatable loading of the drug in the preblend, thereby, requiring minimal amounts of the preblend mixture in the final solid dosage form. Accordingly, a final solid dosage form with a desired drug concentration may be provided which includes dextrates, hydrated at a concentration of less than 0.5 g per serving or dosage unit, thereby qualifying the final solid dosage form as a sugar-free product.

The present invention is further directed to compositions and methods for administering the sugar-free, solid dosage form by oral transmucosal delivery. “Oral transmucosal delivery” refers to the delivery of a pharmaceutical agent across a mucous membrane in the oral cavity, pharyngeal cavity, or esophagus, and may be contrasted, for example, with traditional oral delivery, in which absorption of the drug occurs in the intestines. Accordingly, routes of administration in which the pharmaceutical agent is absorbed through the buccal, sublingual, gingival, pharyngeal, and/or esophageal epithelium are all encompassed within “oral transmucosal delivery,” as that term is used herein. Preferably, oral transmucosal delivery involves the administration of an oral transmucosal solid dosage form to the oral cavity of a patient, which is held in the mouth and dissolved, thereby releasing the pharmaceutical agent for absorption through the tissues of the oral cavity. Inevitably, as the solid dosage form dissolves in the oral cavity, some of the saliva containing the pharmaceutical agent may be swallowed and a portion of the drug may ultimately be absorbed from the intestines. However, the desired therapeutic effect is achieved primarily by the portion rapidly absorbed via the oral mucosa.

As used herein the term “oral transmucosal solid dosage form” broadly refers to any solid delivery form suitable for administering a pharmaceutical agent by oral transmucosal delivery, including patches, troches, lozenges, pastilles, sachets, sublingual tablets, lozenges-on-a-handle, and the like. A preferred form includes patches, lozenges, sublingual tablets, and lozenges-on-a-handle. An especially preferred form is the lozenge-on-a-handle, in which the solid dosage form has a handle affixed thereto. The solid dosage form may be held between the cheek and gum or placed on or under the tongue, or it may be actively licked, sucked, or rubbed across the oral mucosa by the patient or a caregiver. Preferably, the solid dosage form is not bitten or chewed.

Employing the pharmaceutical compositions of the present invention, a pharmaceutical agent may be introduced into the patient's bloodstream almost as fast as through injection, and much faster than using the oral administration route, while avoiding the negative aspects of those delivery methods. The present invention achieves these advantages by incorporating the drug into a dissolvable matrix material. A solid dosage form within the scope of the present invention can be used to administer drugs in a dose-to-effect manner, or until the precise desired effect is achieved. In preferred embodiments, the dosage form has an appliance or handle attached thereto to permit easy removal from the patient's mouth, once the desired effect has been achieved. The presence of the handle facilitates the placement and movement of the dosage form in the mouth, thereby, providing a larger mucosal surface area for absorption of the drug and, if desired, allows the patient or caregiver to conveniently remove the dosage form resulting in the discontinuation of drug administration.

The solid dosage forms of the present invention are sugar-free. As used herein, the term “sugar” refers to mono-, di-, and oligo-saccharides, also known in the art as non-hydrogenated carbohydrates of empirical formula (CH₂O)ₙ, examples of which include glucose, mannose, galactose, ribose, dextrose, fructose, maltose, sucrose, levulose, and lactose. The term “sugar” notably includes saccharides that, when administered by oral transmucosal delivery, may be cariogenic and/or may be metabolized (for example, by hydrolysis or fermentation) to compounds that are cariogenic. For purposes of this application, the terms “glucose” and “dextrose” may be used interchangeably.

The term “sugar” does not include polyhydric alcohols (sometimes referred to as “sugar alcohols” or hydrogenated saccharides), such as sorbitol, mannitol, xylitol, and erythritol, or the sugar derivatives of polyhydric alcohols, such as maltitol, lactitol, isomalt, and polyalditol. The term “sugar” also does not include complex carbohydrates such as gums and polysaccharides, including starch and cellulose, nor their derivatives, such as hydroxy ethyl starch and carboxymethylcellulose. Preferably, the term “sugar” also does not include mono-, di-, and oligosaccharides that are non-cariogenic.

The term “sugar-free” refers to compositions that are mostly free of “sugar,” as defined above. By “mostly free” it is meant that the compositions contain less than about 0.5 grams of sugar per serving, as specified by the U.S. Food and Drug Administration in 21 CFR 101.6(c). For the purposes of the present invention one dosage unit is considered a serving. The sugar-free compositions of the present invention are also mostly free of complex carbohydrates and/or polysaccharides that may be readily converted to sugars in the oral cavity, when the solid dosage form is administered to a patient for oral transmucosal delivery.

For some embodiments, it is important that an oral transmucosal solid dosage form exhibit satisfactory patient-controlled dissolution rates, drug stability, and otherwise be suitable for oral transmucosal delivery. In order to meet these criteria, it is necessary to evenly disperse the pharmaceutical agent in the solid dosage form. In some embodiments of the present invention, a pharmaceutical agent is first mixed with an adherence agent to provide a preblend mixture having a highly uniform concentration of the pharmaceutical agent. The preblend mixture is then added to, and mixed with a pharmaceutically acceptable sugar-free excipient. The final dry powder mixture is compressed into single, solid dosage forms, each individual dosage form having a desired concentration and total quantity of the pharmaceutical agent, and having the agent evenly distributed throughout.

The adherence agent comprises physical features that compatibly receive the high potency pharmaceutical agent during the preblend process. In some instances, the physical form of the adherence agent consists of macroporous granules of ovoid and spheroid shape with a preferred median particle size of 190-220 microns. The adherence agent may further include a rough surface texture with many gaps and pores or other interstitial spaces that are capable of receiving, or being “loaded” with a high potency pharmaceutical agent when mixed together under suitable conditions. In some embodiments, an adherence agent is selected based upon its ability to form a reliably uniform, segregation-resistant preblend with the high potency pharmaceutical agent. In other embodiments, the adherence agent comprises a physical structure having surface features that maximally and compatibly receive the high potency pharmaceutical agent, thereby resulting in high capacity loading of the adherence agent's surface.

The pharmaceutically acceptable sugar-free excipient provides bulk and helps control the dissolution rate of the dosage form in the user's mouth. The term “pharmaceutically acceptable,” as used herein, refers to materials that are generally regarded as safe (GRAS) for use in pharmaceutical products, including when the compositions are administered by the oral transmucosal route, according to methods described herein. The term “patient,” as used herein, refers to humans.

Preferably, the sugar-free excipient is one that will not impart an unpleasant taste to the solid dosage form, such that it might deter a patient from using the product for oral transmucosal delivery. One type of pharmaceutically acceptable sugar-free excipient that is particularly suitable for use in the present compositions, since it meets this requirement, is the class of excipient known as polyhydric alcohols. In particular, the polyhydric alcohols that are commonly used in preparing sugar-free confections, are preferred. Exemplary polyhydric alcohols include, for example, but are not limited to, sorbitol, mannitol, xylitol, erythritol, maltitol, lactitol, isomalt, and polyalditol; and any of the optical isomers and crystalline forms of such polyhydric alcohols as may be used as an appropriate sugar-free substitute. Preferred polyhydric alcohols include xylitol, isomalt, and polyalditol. Various excipients containing these polyhydric alcohols are available commercially. In addition to their natural sweetness, these excipients are particularly suitable because they do not promote the formation of dental caries, i.e., they are said to be non-cariogenic when consumed in accordance with the methods of the present invention. Also they preferably do not lead to an increase in blood glucose, that may be contraindicated, for example, in diabetic patients. These polyhydric alcohols also preferably act as reduced calorie sugar substitutes.

A summary comparison of selected properties of several pharmaceutically acceptable polyhydric alcohols are provided in Table 1, as follows:

TABLE 1
Selected properties of several pharmaceutically acceptable polyols

| | | | Effect | Calorie | Solubility | | |
|---|---|---|---|---|---|---|---|
| | | Relative | on blood | value | in water | | Relative |
| | Derived | sweetness | sugar and | (Kcal/g) | (g/100g) @ | | Hygroscopicity |
| Name | from | (Sucrose 1) | insulin | (Sucrose 4) | 25° C. | 40° C. | (Sucrose Med) |
| Mannitol | Fructose | 0.5-0.7 | Low | 1.6 | 15 | 24 | Low |
| Sorbitol | Glucose | 0.5-0.7 | Low | 2.6 | 66 | 75 | Medium |
| Xylitol | D-xylose | 0.87-1.0 | Low | 3 | 60 | 70 | High |
| Maltitol | Corn syrup | 0.74-0.95 | Low | 3 | 63 | 72 | Medium |
| Lactitol | Lactose | 0.3-0.4 | Low | 2 | 51 | 61 | Medium |
| Isomalt | Sucrose | 0.45-0.65 | Low | 2 | 23 | 40 | Low |
| Erythritol | Glucose | 0.6-0.8 | Low | 0.2 | 37 | 46 | Very low |
| Polydextrose | Dextrose, | 0 | Low | 1 | ~70 | | High |
| | sorbitol & | | | | | | |
| | citric or | | | | | | |
| | phosphoric | | | | | | |
| | acid | | | | | | |

Formulators can select a polyhydric alcohol for their sugar free product based on the characteristics they believe are most preferred for their particular application. For the typical solid oral dosage form intended to be swallowed or chewed, almost any of the above can be used with the choice being made based on one or more physical properties, availability and cost. However, for some embodiments of the present invention the sugar-free bulking agent chosen for the oromucosal lozenge dosage forms requires further additional considerations dictated by their mass, dimensions and morphology, as mentioned previously. These physical requirements, as well as taste, mouth feel, dissolution characteristics and resistance to crumbling in the mouth, present new formulation challenges compared to conventional dosage forms that are intended to be chewed or simply swallowed. Accordingly, selection of these bulking agents requires experimentation and evaluation that exceeds the ordinary skill in the art. The evaluation process for these important additional considerations are described as follows.

Qualification and Selection of the Preferred Sugar-Free Carrier Material or Bulking Agent

Polyhydric alcohols are supplied in different grades to fulfill different needs in the manufacture of food and pharmaceutical preparations. For the purposes of the present invention made by direct compression of dry powder mixtures only the agglomerated or direct compression (DC), grades of polyhydric alcohols are relevant. Compaction trials involving a number of these materials and selected combinations thereof were performed to evaluate their suitability for making the various oromucosal lozenge dosage forms of the instant invention. This evaluation resulted in the identification of an agglomerated grade of isomalt as the preferred carrier material and grade that combines a suite of physical properties (moderate level of sweetness, low solubility, low hygroscopicity and morphology) with superior compaction characteristics and robustness for the intended application.

The evaluation process that resulted in this selection is summarized in Table 2:

TABLE 2
Evaluation of direct compression grade sugar-free carrier/bulking ingredients and combinations
for making oromucosal lozenges of the invention

| Ingredient/ | | Compaction | |
|---|---|---|---|
| combination | Grade/source | properties | Other attributes |
| Mannitol | Spray dried | Good | Surfaces of compressed units are rough or powdery. |
| | Mannogem EZ | | |
| | spray | Poor | Compressed units are soft, friable and crumble in |
| | Pearlitol 200 | | the mouth. |
| Sorbitol | Various suppliers | Good | Most units are hard with matte or shiny surfaces |
| | | | that are slightly tacky to the touch and have a |
| | | | relatively high level of sweetness. Most units |
| | | | dissolve smoothly in the mouth. |
| Xylitol | Xylitab-200 | Poor | Compressed units have rough, powdery, dull |
| | | | surfaces. Units dissolve rapidly with a slight |
| | | | cooling sensation and tend to crumble in the mouth. |
| Lactitol | Finlac DC | Fair to good | Surfaces of compressed units are smooth and shiny |
| | | | upon ejection from the die, but feel rough and |
| | | | crumble in the mouth. |
| Isomalt | Isomalt DC 100 | Good | Compressed units are smooth and shiny on ejection, |
| | | | have low sweetness, and a smooth mouth feel. A |
| | | | binder is needed to avoid delamination. |
| *Polyalditol | Innovatol PD30 | Excellent | Compressed units are smooth, shiny and appear to |
| | | | be very hard. Almost no sweetness, smooth mouth |
| | | | feel and moderate dissolution time in the mouth. |
| | | | Needs an additional sweetener. |
| Mannitol/polyalditol | Mannogem EZ | Fair to good | Compressed units have shiny, smooth surfaces; |
| | spray | | rough mouth feel and low sweetness. Crumbling in |
| | Innovatol PD30 | | the mouth decreases as polyalditol content |
| | Proportions | | increases. None of the units are robust enough for |
| | (M/Inn) | | patient use. |
| | 96/4 | | |
| | 92/8 | | |
| | 84/16 | | |
| Mannitol/sorbitol | Mannogem EZ | Fair | Compressed units have shiny, smooth surfaces; very |
| | spray | | low sweetness, and rough mouth feel. Unit |
| | Sorbitol, various | | hardness increases with sorbitol content. All units |
| | Proportions (M/S) | | crumble in the mouth. |
| | 96/4 | | |
| | 92/8 | | |
| | 84/16 | | |
| Xylitol/sorbitol | Xylitab 200 | Poor | Compressed units have scratched, powdery |
| | Sorbitol P300 | | surfaces; the upper portions are poorly compressed |
| | Proportions (X/S) | | indicating uneven compression. Units have a fairly |
| | 84/16 | | sweet taste. Units with the lowest sorbitol content |
| | 68/32 | | crumble in the mouth. Units with the highest |
| | | | sorbitol content appear to be very hard and |
| | | | delaminate. |
| Isomalt/polyalditol | Isomalt DC 100 | Good | Compressed unit surfaces are smooth and shiny. |
| | Innovatol PD30 | | Slightly sweet taste; approximately 40% delaminate |
| | Proportions | | on ejection. |
| | (Is/Inn) | | |
| | 84/16 | | |
| Isomalt/polydextrose | Isomalt DC100 | Good | Compressed unit surfaces are smooth and shiny. |
| | Litesse Ultra | | Slight sweetness and rough mouth feel. The |
| | Proportions (I/L) | | combination has an ′off′ taste. Polydextrose has |
| | 90/10 | | some, but insufficient binder effect. |
| Isomalt/§hydroxypropyl | Isomalt DC 100 | Good | Compressed units have smooth surfaces, slightly |
| cellulose (HPC) | HPC 95 kDA | | sweet taste and approximately 20% delaminate and |
| | molecular weight | | other units break on ejection. Rough particles of |
| | Proportions | | HPC protrude resulting in an unacceptable bumpy |
| | (I/HPC) | | surface as units dissolve in the mouth. |
| | 98/2 | | |
| Isomalt/sorbitol | Isomalt DC 100 | Good | Compressed unit surfaces are smooth and shiny. |
| | Sorbidex P16656 | | Slightly sweet; slightly rough mouth feel. Dosage |
| | Proportions (US) | | units have a tendency to stick to the bottom punch, |
| | 90/10 | | but there is no breakage or capping. Units dissolve |
| | | | evenly in the mouth. |
| Isomalt/¶polyethylene | Isomalt DC 100 | Excellent | Compressed unit surfaces are smooth and shiny; |
| glycol (PEG) 4000 | PEG 4000 | | slight sweetness and dissolve smoothly in the |
| | Proportions | | mouth. A slight off-color mottling is noted. |
| | (I/PEG) | | |
| | 80/20 | | |
| Isomalt/polyethylene | Isomalt DC 100 | Excellent | Compressed unit surfaces are smooth and shiny. |
| glycol (PEG) 8000 | PEG 8000 | | Units are hard with low sweetness; dissolve |
| | Proportions | | smoothly in the mouth. There is some mottling but |
| | (I/PEG) | | less than with PEG 4000. |
| | 80/20 | | |

*Polyalditol PD30 is a dry form of hydrogenated starch hydrolysate (HSH) consisting of sorbitol, maltitol and hydrogenated polysaccharides. From: Self Gras Determination, submitted by Grain Processing Corporation, Muscatine, IA and SPI Polyols, New Castle, DE, Sep. 11, 2000.
§Hydroxypropyl cellulose (HPC) is a derivatized cellulose commonly used as a binder in solid pharmaceutical formulations.
¶Polyethylene glycols (PEG) are water soluble polymers of ethylene available in various molecular weights denoted by a number in the grade designation. PEGs are widely used in pharmaceutical preparations; one such use is a binder in compressed formulations.

Samples of test materials and combinations without drug or other ingredients were lubricated with magnesium stearate. Individual 2 g aliquots were poured into dies and compressed using a rotary tablet press equipped with tooling to make dosage units of a size, shape and weight envisioned for the invention. It is understood that these particular size, shape and weight parameters do not exclude other possible dosage unit designs. Compressed units were examined visually and sensory properties (smoothness, sweetness, taste, and robustness while dissolving in saliva) were evaluated by placing them in the mouth to simulate conditions of patient use.

As can be seen in Table 2, evaluation of compaction properties coupled with appearance and sensory attributes are necessary to qualify the sugar-free carrier material(s) used in manufacture of the dosage units of the invention. Materials that are commonly used to make perfectly satisfactory smaller compressed dosage forms (e.g., mannitol, sorbitol, xylitol) may have serious deficiencies for dosage units of the present invention. Therefore, substantial additional considerations are required beyond a material's physical characteristics in order to optimize such specialized oromucosal lozenge dosage forms.

While a compressible grade of sorbitol might be considered, its high sweetness level and hygroscopicity are disadvantages. More preferred are combinations of compressible isomalt with its less intense sweetness, smooth mouth feel, low hygroscopicity, lower solubility and smooth dissolution in the mouth. With another ingredient to act as a binder such as sorbitol, polyalditol, or suitable molecular weight grades of PEG, isomalt units are more preferred, while the combination of isomalt and PEG 4000 or 8000 are most preferred.

In some embodiments, isomalt is the preferred polyhydric alcohol primary excipient or carrier used for the sugar-free formulations of the present invention. Isomalt is a disaccharide sugar alcohol derived from sucrose that is suitable for use in pharmaceuticals. It is compendial in the USP-NF, Ph Eur and BP. The components of isomalt are 6-O-α-D-glucopyranosyl-D-sorbitol (1,6-GPS) and 1-O-α-D-glucopyranosyl-D-mannitol dihydrate (GPM), sometimes referred to as gluco sorbitol and gluco mannitol, respectively. Isomalt is available in several grades and is used worldwide in the manufacture of sugar-free confectionery, food and OTC drug products. Isomalt is about half as sweet as sugar, provides approximately half as many calories as sugar and has low glycemic and insulinemic indexes making it suitable for diabetics. Isomalt is noted for its chemical stability and non-reactivity making it compatible with a wide range of active ingredients including fentanyl citrate. Because isomalt is not fermented by cariogenic streptococci in the mouth it is also classified as a non-cariogenic sweetener. Isomalt is thermostable; exhibits low hygroscopicity at 25° C. up to 80% relative humidity, and is highly resistant to enzymatic and acid degradation Like other polyhydric alcohols the structure of isomalt does not contain reducing groups, therefore, it does not undergo the Maillard reaction (non-enzymatic browning) with other ingredients containing amino groups.

A spray-dried agglomerated form of isomalt intended for compressed dosage forms is preferred. Spray drying produces multi-particulate granules or agglomerates that impart good powder flow and compaction characteristics. Suitable materials include commercially available galenIQ™ grades manufactured by PALATINIT GmbH Mannheim, Germany. The manufacturer-supplied physical properties for two such materials are shown in Table 3.

TABLE 3
Physical properties of commercially available compressible grades of isomalt

| Grade | 720 | | | 721 | | |
|---|---|---|---|---|---|---|
| Form | Agglomerated | | | Agglomerated | | |
| Preferred application | Direct compression | | | Direct compression | | |
| Composition | 1:1 GPS/GPM | | | 3:1 GPS/GPM | | |
| Solubility in water | 25 | | | 42 | | |
| (g/100g) | | | | | | |
| Particle size distribution | d10 | d50 | d90 | d10 | d50 | d90 |
| (microns) | 110 | 260 | 460 | 90 | 220 | 360 |

| | | |
|---|---|---|
| Method | Mechanical sieve shaker | Mechanical sieve shaker |
| Specific surface area BET | 0.28 | 0.31 |
| (m²/g) | | |
| Bulk density | 0.5 | 0.5 |
| (g/mL) | | |
| Tapped density n = 1250 | 0.56 | 0.54 |
| (g/mL) | | |
| Hausner factor | 1.12 | 1.07 |
| Indicator of flowability: | | |
| ratio of tapped density to bulk | | |
| density. A value >1.25 = poor | | |
| Carr index | 10 | 7 |
| Indicator of flowability: values | | |
| >25 = poor; <15 = good | | |
| Angle of repose | 33 | 31 |
| (°) | | |
| tan θ reflects the coefficient of | | |
| static friction | | |
| Flowability (orifice d = 6 mm) | 55 | 57 |
| (s/100g) | | |
| Total water—Karl Fischer | 5 | 2.9 |
| (%) | | |
| Hygroscopicity (25° C.) | ~6.5 @70% RH | ~3 @ 70% RH |
| (%) | | |
| Loss on drying | 0.21 | 0.12 |
| (part. vac @ 25° C.) | | |
| (%) | | |

In addition to the polyhydric alcohol primary excipients discussed previously, the pharmaceutical compositions of the present invention may also contain other bulking agents and/or binding agents, including polymeric compounds, complex carbohydrates and their derivatives, and other materials provided that the oral transmucosal solid dosage forms still meet the definition of “sugar-free” cited previously. Examples of other bulking and/or binding agents include, but are not limited to, polydextrose, cellulosic ethers, and polyethylene glycols (PEG). Preferred examples of cellulosic ethers include, but are not limited to hydroxypropyl cellulose, hydroxyethyl cellulose, carboxymethyl cellulose, and hydroxypropylmethyl cellulose, and derivatives and/or salt forms thereof. Polyethylene glycols are commercially available by grades of average molecular weight indicated by the number in the grade designation; preferred examples of polyethylene glycol include, PEG 3350 to PEG 20,000, more preferred are PEG 4000 to PEG 8,000; specifically, and most preferred are PEG 3350, PEG 4000 and PEG 8000.

Suitable excipients for use in the present invention also include non-cariogenic mono-, di-, oligo-, or polysaccharides. The term “non-cariogenic mono-, di-, oligo-, or polysaccharides” refers to saccharide compounds that, when administered by the oral transmucosal route, are not, or are only minimally metabolized to form acids in the mouth that may lead to the formation of dental caries (i.e., are non-cariogenic). The term “minimally metabolized to form acids in the mouth” means that less than about 10%, and more preferably less than about 5% of the non-cariogenic saccharide compounds may be metabolized, when administered to the oral cavity, to form acids that may lead to the formation of dental caries. An example of a non-cariogenic disaccharide polyol is isomalt and an example of a non-cariogenic polysaccharide is polydextrose.

Selection of a High Potency Pharmaceutical Agent

The pharmaceutical compositions of the present invention also contain a pharmaceutical agent. “Pharmaceutical agent” refers to a substance which may be used in connection with an application that is therapeutic or diagnostic in nature, such as in methods for diagnosing the presence or absence of a disease in a patient and/or in methods for the treatment of disease in a patient. As used herein, “pharmaceutical agent” refers also to a substance that is capable of exerting a biological effect in vivo. The pharmaceutical agents may be neutral or positively or negatively charged, or a zwitterion. Examples of suitable pharmaceutical agents include, inter alia, diagnostic agents, pharmaceuticals, drugs, synthetic organic molecules, proteins, peptides, vitamins, and steroids.

Preferably, the pharmaceutical agent of the present invention comprises a high potency drug. High potency drugs are understood to include any pharmaceutical agent having a low effective concentration in the body, or low EC₅₀ value. In some instances, a high potency drug comprises an EC₅₀ value of less than 10. In other embodiments, a high potency drug comprises an EC₅₀ value of less than 1.0. Further, in some embodiments a high potency drug comprises an EC₅₀ value of less than 0.01.

The present invention may further be applied to a variety of drugs affecting the central nervous system. For example, the present invention may easily be utilized in the administration of high potency opioid agonists (such as fentanyl, alfentanil, sufentanil, lofentanil, and carfentanil), opioid antagonists (such as naloxone and nalbuphine), butyrophenones (such as droperidol and haloperidol); benzodiazepines (such as valium, midazolam, triazolam, oxazolam, and lorazepam); GABA stimulators (such as etomidate); barbiturates (such as thiopental, methohexital, pentobarbital, and hexobarbital); di-isopropylphenols drugs (such as diprivan); and other central nervous system-acting drugs such as levodopa, and modafinil. It will be appreciated that other drugs may also be utilized within the scope of the present invention either singly or in combination provided they can be delivered in therapeutically useful amounts via oromucosal absorption.

Preferred embodiments of the present invention comprise a sugar-free solid dosage form including a high potency pharmaceutical agent comprising fentanyl citrate. Fentanyl citrate is the preferred salt form of the high potency opioid drug fentanyl. Chemically fentanyl is N-(1-phenethyl-4-piperidinyl)-N-phenylpropionamide. Fentanyl citrate is a bitter, white crystalline powder with a melting point of 149-151° C. The preferred physical form of fentanyl citrate for use in the formulation is a fine powder milled to a particle size in the micron range and sized by a sieving process to obtain particles of a maximum size of 100 microns. The preferred shape of the milled material viewed under magnification is predominantly granular. The preferable, more preferred and most preferred particle size ranges for milled fentanyl citrate are shown in Table 4:

TABLE 4
Preferred particle size ranges for milled fentanyl citrate powder

| US Standard | | | |
|---|---|---|---|
| sieve size | Preferred | More preferred | Most preferred |
| (microns) | % through | % through | % through |
| 20 | 25 | 30 | 35 |
| 32 | 50 | 60 | 75 |
| 75 | 75 | 90 | 95 |

The fentanyl citrate used in formulations of the present invention meet USP and Ph Eur compendial requirements for identity, moisture content, residue on ignition, heavy metals, residual solvents, impurities, and assay. However, particle size and shape specifications are important additional functional properties that are required to satisfy the specific needs of some embodiments of the present invention. For example, particle size testing is performed on samples of ingredients according to specifications set by the active ingredient manufacturer and possibly again on receipt by the drug product manufacturer.

Content uniformity in dosage forms containing high potency drugs such as fentanyl is critical to avoid dangerous overdosage or therapeutically ineffective under dosage. In the case of the fentanyl citrate used in dosage units of the present invention ranging in dosage strength from 0.2-1.6 milligrams of fentanyl base, an overdosage of even sub-milligram amounts could prove disastrous for patients. During the high energy milling process to produce micron sized particles of fentanyl citrate, sufficient friction can develop in the mill to produce hard, fused particles of the drug substance small enough to escape visual detection but large enough to produce super potent dosage units if they find their way into powder mixtures used to manufacture the product. Since such particles may occur in only a few dosage units in a batch of tens of thousands, testing samples of finished products cannot be solely relied upon to detect them.

Therefore, merely testing samples of the incoming active raw material for particle size distribution against a raw material specification is insufficient to prevent this problem. A preliminary screening of all active ingredients used in a particular product batch must be performed using a sieve of the appropriate mesh size to eliminate the introduction of such particles. Drug products containing tens to hundreds of milligrams per dose will not be adversely affected by containing an occasional particle of active ingredient in the sub-milligram or in some cases even the milligram range. Applying this important consideration in the selection and handling of highly potent actives goes well beyond the usual state of the art practices for less potent drugs.

An additional critical need when incorporating fentanyl citrate powder or other high potency drug substances into the present invention has to do with assuring excellent drug content uniformity in dry powder mixtures. Fine particle size active ingredients tend to be cohesive and form agglomerates comprised of many individual particles as the result of their small size and high surface energy. Current procedures in the art address this issue by either diluting the finely divided active ingredient with a portion of a major excipient to help distribute the active into the bulk of the blend (i.e. geometric blending), or simply by combining all the ingredients in a mixer and relying on the mixing action to deagglomerate and randomly distribute the active. These approaches might work during formulation development at laboratory or pilot plant batch sizes, but will likely fail when scaled up to commercial batch size. A number of commercial scale batches manufactured this way may actually be successful for a time until a drug content uniformity problem arises that results in batch failure or even a product recall.

Avoiding these problems of inconsistency in product quality requires methods that use materials, equipment and procedures that exceed the current skill in the art and that are necessary to assure content uniformity and stability of dry powder formulations of high potency drugs. These methods and procedures are described and discussed in further detail below.

### Selection of the Adherent Agent and the Mixing Process

As discussed previously, low dose high potency drug formulations with poor drug content uniformity are unacceptable from a regulatory stand point and can be dangerous clinically, particularly if excessive drug concentrations or hot spots occur in even a few dosage units. In the particular case of the drug fentanyl, the inadvertent administration and oral transmucosal absorption of a superpotent dose by a patient could result in respiratory depression or death. This potential problem is even more exacerbated for the oromucosal lozenge-type products of the invention for the following reason. Microgram or low milligram amounts of a high potency active ingredient in a 2 g dosage unit of the invention constitute very low drug concentrations in the formulation. In the case of currently approved 2 g fentanyl oromucosal lozenges (Actiq® and others), dosage levels from 200 to 1600 mcg of fentanyl (base) amount to approximately 0.016% to 0.126% by weight concentrations of the active ingredient fentanyl citrate. These extremely small concentrations are in the range typically allowed for impurity levels of less potent drugs in smaller, conventional dosage forms. Therefore, the formulator must achieve a very high level of drug content uniformity in mixtures and in the final dosage forms made from them. This is especially challenging for the present invention wherein dosage units are produced by direct compression of dry powder mixtures.

The ordinary skill in the art teaches that segregation of dry powder particles can be minimized in two basic ways. One is by matching as closely as possible the particle sizes of the active ingredient and the excipients. This is practically impossible with finely divided actives where particle sizes of the drug and excipients differ greatly. The second way is to form granules containing the active and excipients by means of a dry or wet granulation process. There are several techniques for making these granulations including high shear wet granulation, air fluidized powder beds and powder compaction. These processes are time consuming, costly, and require large capital outlays for plant space and equipment. A major disadvantage of using liquids in wet granulations may be the incompatibility of water-unstable actives or other excipients such as buffers and buffer systems that must be kept in the dry state to prevent activation prior to administration to a patient. Other methods of forming dry particle drug ‘coating’ or binding of active ingredients with carrier excipients include mechanofusion; dry impact blending; magnetic-assisted impaction coating; triboelectrification (basically the static electric charging of particles by friction), and dry powder ordered mixing, have been reviewed by Saharan, et al. [Saharan V A, et al. Ordered mixing: mechanism, process and applications. Asian J Pharm Sci 2008; 3(6):240-259.]

Ordered mixing is also referred to by other terms such as: interactive, adhesive, expansive, and regimented, or structured mixing. For purposes of simplicity, the process used in the present invention will be referred to hereafter as either ordered or adherent mixing or mixture.

At its most basic level, ordered mixing occurs when small particles, in this case meaning a maximum size of 100 microns, and a preferred size wherein the majority of drug particles are about 30 microns or less, of the active ingredient are adsorbed (adhered) on the surface of larger carrier particles. Suitable adherent carriers need to be directly compressible, compatible with the active ingredient, have the appropriate morphology, surface characteristics and adequate loading capacity. Formation of an ordered mixture is a two-step process: first, since very small particle active ingredients tend to cling together or agglomerate, they must be broken up as much as possible into much smaller aggregates or ideally into individual particles. These agglomerates are especially characteristic of active ingredients such as fentanyl citrate. Second, the deagglomerated small particles are adhered to carrier particles by the mixing process. Scanning electron microscopy (SEM) studies have confirmed the formation of small particle assemblies on the irregular surfaces of adherent carrier particles as mentioned previously. Ordered or adherent mixing is additionally a method for optimizing the homogeneity of dry powder mixtures as well as preventing the segregation of the active from the adherent carrier material during subsequent manufacturing steps.

Some embodiments of the present invention use a dry powder ordered mixing preblend process that exceeds the ordinary skill in the art and avoids the disadvantages of traditional methods for producing segregation-resistant mixtures of a high potency active and a particular carrier excipient explicitly chosen for that purpose.

In addition to the importance of the adherent carrier excipient selection, the manufacture of effective adherent mixtures also requires attention to the selection of mixing equipment and experimentation to optimize mixing conditions. Not all mixer or blender types are suitable for this purpose. For example high shear mixers may impart too much shear force resulting in breakdown of the granular nature of the adherent excipient, thereby destroying the quality and extent of the interaction between the active and the excipient. The use of an appropriate low shear mixer can avoid this problem. Likewise, the ratio of active to adherent excipient is important to avoid over-loading the interactive capacity of the excipient. Optimizing the volume of the preblend mixing container is another important factor in avoiding the loss of high potency, low dose actives to the container surfaces. Other important factors such as optimizing the mixing container fill volume as a percentage of container capacity, as well as mixing speed and mixing time can be critical to the formation of effective adherent mixtures. These and other considerations are discussed in more detail in the following sections.

The choice of the adherent excipient ingredient and the amount used for producing adherent mixtures is important because optimizing the properties and the quantity of the carrier help assure its capability of sweeping up and holding on to the finely divided drug. It is necessary to avoid overloading the adherent capacity of the carrier material by using a sufficient amount relative to the amount of finely divided active ingredient. This aids in ensuring the formation and stability of the mixture against subsequent segregation. There are excipients that are particularly suited for forming adherent or ordered mixtures. As mentioned previously, these have certain properties, including: inertness and compatibility with actives; surface roughness or rugosity; surface pores and clefts of sizes that can accept and hold deagglomerated fine particles; are acceptable for use in pharmaceutical products; have an appropriate moisture content, and are suitable for direct compression of dosage forms. For example, increasing the moisture content of the adherent excipient, if not incompatible with the active ingredient, is said to increase the stability and segregation resistance by virtue of an in situ ‘wet’ granulation effect without the addition of liquid water.

Some embodiments of the present invention comprise Emdex® as the adherent agent or excipient for the formation of ordered mixtures of the invention. Emdex is a commercial form of dextrates, hydrated well established as a useful carrier material for producing stable adherent mixtures with finely divided particles. Emdex consists of spray crystallized dextrose (DE 93%-99%) and minute amounts of starch oligosaccharides from plant sources. The physical form is crystallized macroporous granules of ovoid and spheroid shape with a preferred median particle size of 190-220 microns. The adherent excipient consists of many randomly arranged flat microcrystals bound together by very small amounts of higher saccharides and interspersed with variously shaped void spaces that serve as contact points for finely divided active drug substances and is intended for use in tableting by direct compression. Under high magnification the granules exhibit a rough surface texture with many gaps or pores. Reported interparticulate pore volume is approximately 0.6 mL/g and typical surface area values are 1000-1200 cm²/g [Staniforth JN. U.S. Pat. No. 4,349,542, 1982]. When mixed with finely divided active ingredients of less than 100 micron particle size, these granular particles are capable of producing adherent mixtures with excellent content uniformity i.e., that have coefficients of variation of drug content between samples of 5% or less. The presence of very small particles of dextrates, hydrated will diminish the effectiveness of forming adherent mixtures and very large particles may contribute to interparticle segregation when mixed with other powders, therefore, the particle size range must be controlled. These problems are avoided in the manufacture of products of the invention by removing fines less than about 200 microns and any large clumps and particles greater than 600 microns by passing the material through appropriate mesh size screens prior to use.

Bioequivalency

Patients using prior art sugar-based oral transmucosal solid dosage forms may have already become accustomed to the rate of onset and the extent of the drug effect that may be achieved from using such products. Where the drug exerts a potent effect on the central nervous system, as in the case of fentanyl for example, it may be vital that the sugar-free solid dosage form have a bioavailability that is similar to a sugar-containing solid dosage form lest overdosage result. Thus, it is preferred that a sugar-free oral transmucosal solid dosage form of the present invention be bioequivalent to a sugar-containing oral transmucosal solid dosage form. “Bioequivalent”, as used herein, refers to the standard applied by the respective national regulatory agency in a country for which marketing approval of the invention is sought. For example, for a composition of the invention to be bioequivalent in the United States, it must comply with the definition of bioequivalence as defined by the U.S. Food and Drug Administration in 21 CFR 320.1. Similarly, two solid dosage forms are considered bioequivalent, as that term is used herein, if the rate and extent of absorption of the pharmaceutical agent present in the dosage forms are not significantly different, when administered to human subjects at the same molar dose under similar experimental conditions.

Additional Additives

In addition to the high potency pharmaceutical agent, the primary excipient, and the adherent excipient, the sugar-free compositions of the present invention may also contain optional ingredients, such as flavorings, sweeteners, flavor enhancers, releasing or lubricating agents, and pH buffers. All of these inactive ingredients should preferably be on the GRAS list, to assure that they are pharmaceutically acceptable. Alternatively, an inactive ingredient should be self-proclaimed GRAS or, at least, acceptable for use in food.

It may be desirable to add a flavoring agent to the compositions of the present invention. A wide range of flavors are available for preparing good tasting and desirable medications within the scope of the present invention. These may be required in order to mask the unpleasant taste of the drug. Flavorings may be combined, as desired, to produce a particular flavor mix which is compatible with a particular medication. Some of the confectioner's flavorings which may be used in the context of the present invention include artificial vanilla, vanilla cream, mint, berry, cherry, spearmint, grape, coconut, chocolate, menthol, licorice, lemon, and butterscotch. Each of these flavorings is obtainable in a concentrated powder form. Flavoring agents prepared by spray drying are most preferred. Other flavorings known in the confectionery arts may also be acceptable because of the ease of combining the ingredients of the present invention. Any number of flavorings may be combined in any desired ratio in order to produce the specific desired taste characteristics required for any particular application. For example, flavor combinations may be varied in order to be compatible with the flavor characteristics of any specific drug.

In order to produce a desirable color for the end product, artificial colorings may also be added to the composition. The flavorings described above are generally a white or off white powder, as are the other major components. Therefore, additional coloring is necessary if a colored end product is desired. Coloring may also be important as a code to indicate the type and concentration of drug contained within a particular lozenge-on-a-handle. Any type of color known to be safe, and thus generally used in the confectionery trade, or otherwise approved by the appropriate regulatory authority for use in pharmaceutical preparations, may be used to provide coloring to the product.

In order to provide a good tasting medication, it may be necessary to add additional sweeteners to the composition. Since the compositions are sugar-free, an artificial sweetener, such as aspartame, acesulfame K, saccharin, sucralose, altitame, cyclamic acid and its salts, glycerrhizinate, dihydrochalcones, thaumatin, monellin, or any other non-cariogenic, sugar-free sweetener may be used, alone or in combination. For compositions which contain a sugar alcohol based excipient, additional sweeteners may not be necessary, due to the naturally sweet taste of these polyhydric alcohols. Again, it is desired that a sweetener or combination of sweeteners be obtained which is compatible with the pharmaceutical agent and the other components such that a good tasting solid dosage form is produced. For some applications, it may be desirable to add a flavor enhancer to the composition in order to achieve a good tasting product. Flavor enhancers provide a more pleasant sensation in the patient's mouth during oral transmucosal administration. Flavor enhancers within the scope of the present invention include materials such as ribotide (a nucleotide) and monosodium glutamate (MSG). Other flavor enhancers are known to those of skill in the art.

Additional excipients in the oromucosal lozenges of the invention may comprise other ingredients commonly used in compressed dosage forms, including but not limited to binders such as natural starches, gelatin, tragacanth, derivatized celluloses, polymers such as polyethylene glycols, polyvinyl alcohols, and polyvinyl pyrrolidone. Formulations containing ionizable active ingredients may also contain physiologically acceptable acidifying or alkalizing compounds or buffer systems to produce the optimal pH conditions for oral transmucosal absorption of the active ingredient. Since the products of the invention are intended to be dissolved slowly in the mouth, the extended residence times in the buccal cavity may require the inclusion of ingredients to mask unacceptable taste or otherwise enhance the sensory profile of the drug product for patient acceptability. Such ingredients may include additional pharmaceutically acceptable artificial sweeteners and flavors or colors previously mentioned.

A lubricant is usually required for the manufacture of compressed solid dosage forms for the purpose of lubricating the tablet press tooling. Multiple lubricant types may include but are not limited to the hydrophobic agents such as magnesium stearate, calcium stearate, stearic acid or sodium stearyl fumarate, or water soluble agents such as the polyethylene glycols (PEG) or sodium lauryl sulfate. In addition to lubricants, other materials known as glidants may also be beneficially used depending on the needs of the specific formulation to improve powder flow, aid in ejection of products from the tablet press or in some cases, to prevent loss of the active ingredient by adherence to surfaces of the manufacturing equipment. Glidants may include acceptable grades of talc and fumed silicon dioxide. The amounts and grades of these other ingredients are selected based on the needs of the particular product being manufactured.

Oromucosal lozenges of the invention with a holder may also require the use of an edible glue to affix a suitable holder to or into the compressed powder dosage form. The ingredients for such a glue typically consist of food grade starch, a sugar and water (removed on drying). The edible glue, if used, contributes a minute amount (less than 50 mg) of simple sugar to a finished dosage unit and thereby does not invalidate the sugar-free status of the product as defined by regulation.

Some embodiments of the present invention may further include a permeation enhancer to assist the pharmaceutical agent in diffusing across the mucosal membrane. Typical permeation enhancers may include bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate. Other permeation enhancers such as sodium dodecyl sulfate (“SDS”), dimethyl sulfoxide (“DMSO”), sodium lauryl sulfate, salts and other derivatives of saturated and unsaturated fatty acids, surfactants, bile salt analogs, derivatives of bile salts, or such synthetic permeation enhancers as described in U.S. Pat. No. 4,746,508 and U.S. Pub. App. No. 2004/0253307, the disclosures of which are incorporated herein by reference in their entireties, may also be used. It is generally believed that bile salts are good enhancers for hydrophilic drugs and long chain fatty acids, their salts, derivatives, and analogs are more suitable for lipophilic drugs. DMSO, SDS, and medium chain fatty acids (about C-8 to about C-14) their salts, derivatives, and analogs may work for both hydrophilic and lipophilic drugs.

The permeation enhancer concentration within the dissolvable matrix material may be varied depending on the potency of the enhancer and rate of dissolution of the dissolvable matrix. Other criteria for determining the enhancer concentration include the potency of the drug and the desired lag time. The upper limit for enhancer concentration is set by toxic effect to or irritation limits of the mucosal membrane.

### Undesirable Ingredients

Pharmaceutical products intended to be swallowed typically contain a disintegrant to aid in the release of the active ingredient in the GI tract. By contrast, the products of the invention are intended to dissolve relatively slowly in the patient's mouth by a process of surface erosion. Therefore, the use of disintegrants is undesirable and it is specifically necessary to exclude any ingredients from formulations that may swell when in contact with aqueous media and cause disintegration of the dosage forms. Notwithstanding the examples of other excipients mentioned previously, all must be evaluated for their tendency to promote disintegration and those that do must be excluded from formulations.

### Methods of Manufacture

Specific methods and processes are required to make the invention. The methods and processes are the result of substantial product development research to determine quantities and grades of materials, mixing equipment and mixing parameters, in-process testing, compression equipment and conditions, post-compression operations and packaging materials and methods.

A schematic of the general process for manufacturing dosage units of the present invention is shown in Table 5, as follows:

TABLE 5
Summary of the manufacturing process

| Step | Tests |
|---|---|
| Qualification of raw materials | Release and particle size testing and sieving (as necessary) of |
| | the active ingredient and key excipients |
| Preparation of adherent mixture preblend | Assay content uniformity of the preblend. Testing may be |
| | discontinued after process is validated during development. |
| Main dry powder blend | In-process assurance of blend content uniformity |
| | (unit dose samples) |
| Compression | In-process dosage unit content uniformity, assay, weight, |
| | hardness, friability, in-vitro dissolution, etc., as required by |
| | product specifications |
| Assembly (if required) | Holder-dosage unit integrity, if applicable |
| Packaging | Final release testing as required by specification |

### Qualification and Release of Raw Materials

All raw materials intended for the manufacture of commercial products are subjected to testing and release procedures to assure compliance with pharmacopeial requirements and meet other functional requirements of each product of the invention.

### Preparation of Adherent Mixture Preblend

There are several approaches for incorporating a small amount of powdered material such as a high potency active ingredient into a much larger volume bulk mixture. The processes of wet and dry granulation have been mentioned previously. Another traditional method is known as geometric dilution. In this procedure approximately equal amounts of the active ingredient and a carrier material are mixed together, followed by additional portions of the carrier approximately equal to the previous amount of mixture until all the carrier and other formulation ingredients have been added. This description of geometric mixing is offered as an example of a traditional method that is not suitable for the purposes of this invention because of its multiple steps and its time and labor intensity.

Alternatively, the use of preblends is an accepted way to incorporate small quantities of powdered ingredients into the bulk mixture. In the case of finely divided active ingredients, preblending with a suitable carrier is an effective way to (1) deagglomerate the particles, (2) densify the material so that it does not ‘float’ on the larger particles in the mixture, and (3) distribute the drug particles evenly over the surface of the larger adherent excipient particles to form the adherent mixture of the invention. In the case of OTFC the active ingredient preblend is prepared by combining the milled fentanyl citrate with the appropriate measured quantity of dextrates, hydrated and blended in a sealed container using a low shear mixer such as a Turbula® or other tumble or rotating bin blender at a specified rotational speed for a specified time. A preferred mixing time for forming the adherent preblend is 40-60 minutes. The preferred mixing speed is 40 revolutions per minute. These stated preferred conditions are not to be construed as absolute but are intended as a practical guide in teaching the implementation of the present invention. Shorter or longer mixing times, slower or faster mixing speeds and different blender types may produce acceptable results or may be required depending on any number of circumstances and the needs of specific formulations.

As mentioned previously, low shear mixing is preferred to prevent the breakdown of the granular nature of the adherent carrier and the three-dimensional motion of the Turbula or other Schatz motion blender is most preferred. The Turbula mixer is noted for exposing the materials being mixed to a continually changing three-dimensional rhythmic, pulsing motion consisting of rotation, translation (motion along a linear axis without rotation, e.g., forward, back, up, down, right, left), and inversion in a revolving vessel. Turbula or other Schatz motion mixers are advantageously used for homogeneous mixing of powdery substances having different specific densities and particle sizes such as those used in the manufacture of the present invention.

Attention to the following parameters is important in the formation of an active-adherent carrier preblend in producing a homogeneous mixture: exploiting the morphology and particle size of the active ingredient and a compatible adherent carrier excipient; the ratio of drug to carrier; the fill volume of the mixing container; mixing time, and mixing speed. Of the preceeding, the more important parameters are the nature of the carrier material, as discussed previously in the section on selection of the adherent preblend excipient, the weight ratio of the active ingredient to the carrier, and the particle size of the active. The mixing container fill volume, mixing speed and mixing time are dependent in the following way: all other things being equal the larger the fill volume as a percentage of mixing container capacity, the longer the mixing time required at a given mixing speed for the formation of a homogeneous adherent mixture.

Observing appropriate limits on the ratio of active ingredient to adherent carrier excipient is necessary to prevent overloading the capacity of the carrier material's contact sites. In the case of dextrates the preferred ratio of an active ingredient of a particle size range less than 100 microns is less than or equal to 2% on a weight basis. A more preferred ratio of active ingredient to dextrates, hydrated is less than or equal to 1% on a weight basis and the most preferred ratio is less than or equal to 0.5% on a weight basis. These preferred ratios are not to be construed as absolute and do not rule out variations otherwise capable of producing acceptable results. The maximum amount of a sugar-based carrier excipient that can be used is determined by the amount that does not exceed the quantity allowed by regulation in a sugar-free finished product (0.5 g/unit).

During the product development stage it is useful to test an appropriate number of individual samples of the preblend to validate drug assay and content uniformity. It may also be desirable to subject samples of the preblend to segregation testing to evaluate the quality of the adherent mixture. This type of testing may employ specialized equipment designed to produce sifting and/or air fluidization segregation of powders. Segregation test devices are commercially available, alternatively a stack of sieves and a sieve shaker may be used to test for segregation resistance of a mixture. Well formed adherent or ordered mixtures will be resistant to segregation forces imposed by the test. Mixtures exhibiting low segregation tendency will have drug concentration values with low coefficients of variation (CV) (sometimes referred to as relative standard deviation, RSD). Variation values will preferably be 5% or less and more preferably 2% or less.

### Preparation of the Main Dry Powder Blend

The adherent preblend mixture of the active ingredient and dextrates, hydrated is added to the other ingredients comprising the bulk of the formulation, less the lubricant, preferably to a mixing container of a low shear mixer. An appropriate fill volume of the container is observed such that the bulk ingredients plus the preblend do not exceed a preferred 80% fill volume; a 70% fill volume is more preferred for efficient mixing action and to minimize mixing time. These preferred fill volumes are not intended as strict limits to the exclusion of other volumes that result in acceptable preblend mixtures.

### Manufacture of Compressed Powder Dosage Units

After the addition and blending of the lubricant, the final blend is transferred to the hopper of a tablet press preferably by means of an intermediate blend container (IBC). The tablet press may require special design modifications in order for it to be capable of making compressed dosage units of the shape and size required by the invention. Such a tablet press will have deep fill capability such that a sufficient volume of powder mixture can be accommodated in the tooling dies. Other modifications of the tablet press such as the design of the feed frame and feed frame paddles, and a specially designed hopper to ensure mass powder flow may be required. Compression studies using a direct compression grade of isomalt dry powder formulation produced 2 g dosage units of the invention with acceptable hardness (about 30-50 kilopond (kP), the same as 30-50 kilogram-force (kgf), and low friability (0-0.1%) using tableting speeds of 25,000 or 45,000 tablets per hour and compression forces ranging from about 10 to about 18 kilonewtons (kN), or about 1020 to about 1835 kgf. Other physical property parameters may be acceptable depending on specific product requirements.

### Additional Manufacturing Steps

In some embodiments, additional manufacturing steps may be required depending on the design of the finished product. For example if a holder is to be affixed to the compressed powder dosage unit, the material comprising the holder is required to be radiopaque as well as a method and suitable equipment for assembly of the holder and the dosage unit.

### EXAMPLES

The invention is further demonstrated in the following Examples. The Examples are for purposes of illustration and are not intended to limit the scope of the present invention.

The following examples are provided for a sugar free oromucosal lozenge dosage form of the invention for the drug fentanyl. As discussed previously, this product fulfills the criteria for a low dose, high potency drug. The currently available sugar-based commercial product (Actiq and others) is made in dosage strengths of 200 mcg, 400 mcg, 600 mcg, 800 mcg, 1200 mcg and 1600 mcg of fentanyl base. The active ingredient is the milled form of the particle size specified in the materials section and prescreened for the removal of any large hard aggregates or fused material from the milling process.

### Example 1

### Quantitative Compositions in Six Dosage Strengths

The quantitative compositions of the six dosage strengths are displayed in Table 6. All ingredient amounts are in milligrams.

TABLE 6
Quantitative compositions of six sugar-free OTFC dosage strengths

| | | 200 | 400 | 600 | 800 | 1200 | 1600 |
|---|---|---|---|---|---|---|---|
| Ingredient | Function | mcg | mcg | mcg | mcg | mcg | mcg |
| Fentanyl | Active | 0.3142 | 0.6284 | 0.9426 | 1.2568 | 1.8852 | 2.5136 |
| citrate* | ingredient | | | | | | |
| dextrates, | Adherent | 63 | 126 | 189 | 252 | 378 | 440 |
| hydrated | mixture | | | | | | |
| | excipient | | | | | | |
| Isomalt | Bulk | 1655.7 | 1592.4 | 1529.1 | 1465.8 | 1339.2 | 1276.5 |
| | carrier | | | | | | |
| | excipient | | | | | | |
| Polyethylene | Binder | 191 | 191 | 191 | 191 | 191 | 191 |
| glycol 8000 | | | | | | | |
| Citric acid | Buffer | 12 | 12 | 12 | 12 | 12 | 12 |
| anhydrous | component | | | | | | |
| Dibasic | Buffer | 28 | 28 | 28 | 28 | 28 | 28 |
| sodium | component | | | | | | |
| phosphate | | | | | | | |
| anhydrous | | | | | | | |
| Artificial | Flavor | 30. | 30 | 30 | 30 | 30 | 30 |
| flavor | | | | | | | |
| Magnesium | Lubricant | 20 | 20 | 20 | 20 | 20 | 20 |
| stearate, | | | | | | | |
| non-bovine | | | | | | | |
| Total | | 2000 | 2000 | 2000 | 2000 | 2000 | 2000 |
| (rounded) | | | | | | | |

*Fentanyl citrate amounts reflect a molecular weight ratio of the citrate salt to fentanyl base of 1.571. Therefore, the amount of fentanyl citrate required for each dosage strength = 1.571 × the label strength of fentanyl base.

The formulations shown in Table 6 include a binder ingredient necessary to manufacture compressed dosage units of a specific shape, a buffer system to control pH, and a flavor ingredient. The bulk of the dosage units consists of the sugar-free carrier material isomalt. The differences in composition between the dosage strengths are the amounts of fentanyl citrate and dextrates, hydrated. The compensation for different amounts of these two ingredients is made by varying the amount of isomalt. The amounts of dextrates, hydrated in each formulation are selected to keep the ratio of active ingredient to dextrates, hydrated constant at approximately 0.5%-0.6% to avoid overloading the adsorptive capacity of the adherent excipient. The flavor is food grade and all other ingredients are compendial in one or more of the major pharmacopoeia. Isomalt is direct compression grade and dextrates, hydrated is specifically made for use in direct compression applications.

### Example 2

### Special Considerations for Fentanyl Citrate Products

### 1. Preparation of the Adherent Mixture Preblends

The general procedure for making the adherent mixture pre-blends was described previously. The compositions of the sugar-free OTFC product examples illustrate an important consideration to be made in making the preblends, as follows:

The amount of dextrates, hydrated used to prepare the adherent mixture preblends for the example formulations in Table 6 above varies as shown in Table 7. The amounts of fentanyl citrate and dextrates, hydrated are for a batch size of 140 kg.

TABLE 7
Weight of fentanyl citrate & volume of dextrates, hydrated in the
example preblends

| | | | *Approximate |
|---|---|---|---|
| | | | volume of |
| | Fentanyl | dextrates, | dextrates, |
| Dosage | citrate | hydrated | hydrated |
| strength | (kg) | (kg) | (Liters) |
| (mcg) | Rounded | Rounded | Rounded |
| 200 | 0.02 | 4.41 | 6.5 |
| 400 | 0.04 | 8.82 | 13 |
| 600 | 0.07 | 13.23 | 19.5 |
| 800 | 0.09 | 17.64 | 25.9 |
| 1200 | 0.13 | 26.46 | 38.9 |
| 1600 | 0.18 | 30.8 | 45.3 |

*Based on a bulk density of 0.68 kg/L

The relatively small amounts of fentanyl citrate in the preblends, ranging from approximately 0.022 kg to approximately 0.176 kg, will have a very small effect on the overall volume of the preblends compared to the amount of dextrates, hydrated. The preblend volume is an important consideration because it determines the sizing of the mixing container. Turbula or other Schatz motion mixers produce the most efficient dry powder mixing action when the mixing container is filled to 50-70% of capacity. Observing these limits on mixing container fill minimizes the potential for loss of the active ingredient by clinging to the inner surfaces of the container. This may be especially important for lower dosage strengths where the loss of even small amounts of a high potency active to the surfaces of manufacturing equipment will have a disproportionately large effect on the active ingredient assay in the preblend. Therefore, the optimal capacities of mixing containers for making preblends for these example batches ranges from a maximum of about 13 liters to about 90 liters.

### 2. Unexpected Effects on the Predicted Bioavailability of Different Fentanyl Formulations

In some embodiments, it is important for the product of the invention to be bioequivalent to the sugar-based reference product. Demonstrating bioequivalence of drug products requires a clinical study in humans. Such studies are time consuming and expensive.

Therefore, it is extremely useful for drug product developers to have a reliable surrogate test that can be used to assess drug bioavailability and thereby limit the number of clinical studies that may be required. Such a laboratory surrogate known to and used by those skilled in the art is the in vitro dissolution test. The in vitro dissolution results obtained from reference and test products are compared to help guide development efforts toward achieving a bioequivalent product. This approach is often used for drugs such as fentanyl citrate that are classified as having high solubility and high tissue permeability and has been used in the development of a compressed dry powder sugar free fentanyl citrate oromucosal lozenge.

The in vitro dissolution results for the sugar-free product demonstrated significantly faster release of fentanyl compared to the sugar-based reference product (Actiq) as shown in Table 8.

TABLE 8
In vitro dissolutionprofiles of bioequivalent fentanyl citrate
oromucosal lozenges

| | Units | Mean & standard deviation % fentanyl released | | | | | |
|---|---|---|---|---|---|---|---|
| | tested | by time (min) | | | | | |
| Type | (n) | 5 | 10 | 20 | 30 | 40 | 100 |
| Actiq | 18 | 31.41 | 46.94 | 66.79 | 79.39 | 88.59 | 97.60 |
| (sugar- | | (2.94) | (11.25) | (14.38) | (11.66) | (7.49) | (1.20) |
| based) | | | | | | | |
| Sugar- | 6 | 58.92 | 92.27 | 102.53 | 102.64 | 103.06 | 103.28 |
| free | | (3.44) | (3.37) | (2.19) | (2.64) | (2.16) | (2.47) |
| (Isomalt) | | | | | | | |

Examination of the data in Table 8 clearly shows the substantial difference in fentanyl release rates between the reference (Actiq) and test (sugar-free) products when subjected to the same test conditions. The sugar-free product released more fentanyl at every time point and was completely dissolved within 20 minutes, while the reference sugar-based Actiq required 100 minutes to completely dissolve. These results suggested that the fentanyl bioavailability from the sugar-free test product might be unacceptably high compared to that of the reference and thereby fail to be bioequivalent.

Conventional wisdom teaches that dramatic differences in the in vitro dissolution characteristics of two different formulations of the same drug and dose administered by the same route of administration will not be bioequivalent when administered to patients. To those of ordinary skill in the art these results would typically be sufficient justification for dropping the development of the test formulation and going back to the drawing board with the objective of matching the dissolution profile of the reference product. Therefore, the current skill in the art actually teaches away from the products of the invention based on the grounds that they would very likely not be bioequivalent to the reference and thereby fail to meet a critical product development objective. Unexpectedly, the two products depicted in Table 8 were subsequently shown to be bioequivalent when tested in humans.

A second unexpected effect of these results is the new teaching provided by the present invention with respect to the OT bioavailability of fentanyl. The evidence for this is the convincing contradiction of the conventional wisdom of those of ordinary skill in the art regarding the validity of in vitro dissolution test for this purpose. The basis for this new teaching is the recognition that there may be formulation dependent variables that are more important than product dissolution rate in determining the bioavailability of a drug administered by this route.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What we claim is:

1. A pharmaceutical composition, comprising:
a pharmaceutically acceptable sugar-free excipient; and an adherent, segregation-resistant preblend mixture comprising:
a highly potent active pharmaceutical agent; and
a carrier excipient.

2. The composition of claim 1, wherein the highly potent active pharmaceutical agent is fentanyl citrate.

3. The composition of claim 1, wherein the composition is bioequivalent to a commercially approved sugar-based product.

4. The composition of claim 1, wherein the carrier excipient is an adherent carrier excipient.

5. The composition of claim 4, wherein the adherent carrier excipient is dextrates, hydrated.

6. The composition of claim 1, wherein the pharmaceutically acceptable sugar-free excipient is isomalt.

7. The composition of claim 6, wherein the composition is used for the treatment of pain in humans.

8. A method for manufacturing the pharmaceutical composition of claim 1, the method comprising steps for:
providing an adherent preblend mixture consisting essentially of a high potency pharmaceutical agent and dextrates, hydrated;
mixing a portion of the preblend mixture with a pharmaceutically acceptable sugar-free excipient; and
forming a dosage form of the pharmaceutical composition having a desired concentration of the high potency pharmaceutical agent and a final concentration of the dextrates hydrated that is less than or equal to 0.5 grams per dose or serving.

9. The method of claim 8, wherein the step of providing the adherent preblend employs low shear mixing equipment.

10. The method of claim 8, further comprising a step for in-process testing of the pharmaceutical composition to assure an acceptable level of segregation-resistance of the high potency pharmaceutical agent and the dextrates, hydrated.

11. The method of claim 8, wherein the dosage form comprises a direct compression dry powder solid sugar-free pharmaceutical dosage form.

12. The method of claim 8, wherein the dextrates, hydrated is Emdex.

13. The method of claim 8, wherein the desired concentration of the high potency pharmaceutical agent is from approximately 200 mcg to approximately 1600 mcg per dosage unit.

14. The method of claim 8, wherein the desired concentration of the high potency pharmaceutical agent is from approximately 0.016% to 0.126% by weight concentration in the pharmaceutical composition.

15. The method of claim 8, wherein the dextrates, hydrated comprise a median particle size from approximately 190 microns to approximately 220 microns.

16. The method of claim 8, further comprising a step for adding an artificial sweetening agent to the preblend mixture and the pharmaceutically acceptable sugar-free excipient.

17. The method of claim 16, wherein the artificial sweetening agent is selected from the group consisting of aspartame, acesulfame K, saccharin, sucralose, altitame, cyclamic acid and its salts, glycerrhizinate, dihydrochalcones, thaumatin, and monellin.

18. The method of claim 8, further comprising a step for adding additional ingredients commonly used in compressed dosage forms.

19. The method of claim 18, wherein the additional ingredients are selected from the group consisting of natural starches, gelatin, tragacanth, derivatized celluloses, polymers such as polyethylene glycols, polyvinyl alcohols, and polyvinyl pyrrolidone, flavorants, colorants, magnesium stearate, calcium stearate, sodium stearate, Compritol 888, stearic acid, sodium stearyl fumarate, polyethylene glycol, sodium lauryl sulfate, talc, fumed silicon dioxide, sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate.

20. A sugar-free pharmaceutical composition comprising an oral transmucosal solid dosage form comprising an adherent, segregation-resistant preblend mixture including a highly potent pharmaceutical agent having a particle size of less than 100 microns and an appropriate amount of a suitable adherent carrier excipient.
